(19) United States
(12) Patent Application Publication
(10) Pub. No.: US 2008/0281395 A1
(43) Pub. Date: Nov. 13, 2008

(54) RATCHETING BIO CELL DESIGNS

(75) Inventors: Tracee Eidenschink, Wayzata, MN (US); Todd Bethel, Lakeville, MN (US); Thomas E. Broome, Prior Lake, MN (US); Gordon J. Kocur, Lino Lakes, MN (US); Jens Hegg, Minneapolis, MN (US); Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 11/745,091

(22) Filed: May 7, 2007

(51) Int. Cl. A61F 2/06

(52) U.S. Cl. 623/1.11; 623/1.15

(57) ABSTRACT

A bifurcated stent having a side branch constructed out of a bioabsorbable material. The side branch comprises one or more bio cells which are flexible and easily deformable when the stent is in the unexpanded state. The bio cells have a locking member which fixedly holds the bio cells in place once the stent assumes the expanded state. The locking member uses oppositely directed hooks or ratchet teeth to prevent contraction in the bio cells once the expanded state is assumed. By having an appropriate alternation between flexibility and rigidity the side branch is flexible enough to deal with the geometric difficulties of bending and deploying a side branch while being fixed and rigid enough to properly support and scaffold a side branch assembly. The deployment of the side branch can be facilitated by the use of a ratcheting device. The side branch can also have a flared shape.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices specifically stents and their components.

2. Description of the Related Art

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable). Stents may be implanted to prevent restenosis following angioplasty in the vascular system.

A complication arises when stenoses form at vessel bifurcation sites. A bifurcation site is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

This invention includes a number of embodiments where any one, any combination of some, or all of the embodiments can be incorporated into a stent and/or a stent delivery system and/or a method of use. The present invention is directed to a bifurcated stent in which at least a portion of the bifurcating side branch assembly is constructed out of a bioabsorbable material. This side branch assembly also has ratchet struts and hooking bio cells which facilitate the extension, flaring, and deployment of the side branch lumen. This side branch assembly can be self expanding, expandable by a stent expanding balloon, expandable by a separate side branch balloon, and/or expandable by a leader strip.

At least one embodiment of the invention is directed to a bifurcated stent having an unexpanded state and an expanded state, comprising a generally tubular wall which defines a first lumen and a side branch deploying portion. The side branch portion is constructed of at least one bioabsorbable material engaged to the generally tubular wall. When in the expanded state, the side branch deploying portion extends obliquely outward from the generally tubular wall and forms a side branch which defines a second lumen in fluid communication with the first lumen. The side branch deploying portion comprises one or more bio cells, having a perimeter which defines an opening in the side branch deploying portion, a luminal end at the portion of the perimeter closest to the tubular wall, a radial end at the portion of the perimeter farthest from the tubular wall, and a locking mechanism. The locking mechanism imposes on the bio cell locked and unlocked configurations. When the bio cell is in the locked configuration, the luminal and radial ends are restrained from moving closer to each other.

At least one embodiment of the invention is directed to a bifurcated stent in which the side branch assembly is at least partially defined by one or more obliquely bending petal members positioned near a matrix of bio cells which encircles at least a portion of the side branch. The matrix can extend along that portion of the side branch located where the tubular wall and the side branch are engaged to each other. The matrix can be arranged according to a circular pattern and/or in a configuration in which at least two bio cells on opposite sides of the circular pattern have different expansive capacities. The walls of at least two bio cells on opposite sides of the circular pattern can have perimeters with different overall lengths.

The locking mechanism can comprise at least two shafts each of which extends between a position along the perimeter of a tetrahedral bio cell and an engaging member. The engaging members can become engaged to each other when the shafts are moved into contact with each other as the stent enters into the expanded state. The engaging members can be oppositely directed hooks and when engaged can allow for radial motion but prevent luminal motion. The bio cell can be further braced by side struts and can span between rings. The side branch can also comprise ratcheting mechanisms and can have a flared configuration. These and other aspects of the invention are set forth below.

### BRIEF DESCRIPTION OF THE SEVERAL VIEWS OF THE DRAWINGS

The invention is best understood from the following detailed description when read in connection with accompanying drawings, in which:

FIG. 1 is a schematic perspective view of an expanded bifurcated stent in which the bifurcation comprises a plurality of bio cells.

FIG. 2 is a schematic perspective view of an expanded bifurcated stent in which the bifurcation is flared and comprises a plurality of bio cells.

FIG. 3 is a schematic perspective view of an expanded bifurcated stent in which the bifurcation comprises a plurality of petal members which are engaged to a bio cell matrix at the base of the side branch assembly.

FIGS. 4A-8B are lateral views of some bio cells.

FIG. 9 is an overhead view of an unexpanded ring type side branch assembly in which the rings are interconnected by bio cells bracketed by curved connecting struts.

FIG. 10 is a close up schematic perspective view of an expanded bifurcated stent in which the bifurcation comprises rings interconnected by bio cells, is at least partially extended by a radially directed ratcheting mechanism, and is flared by a circumferentially directed ratcheting mechanism.

FIG. 11 is a lateral view of an unexpanded radially directed ratcheting mechanism.

FIG. 12 is a lateral view of an expanded radially directed ratcheting mechanism.

FIG. 13 is a perspective view of an unexpanded circumferentially directed ratcheting mechanism.

FIG. 14 is a lateral view of an expanded circumferentially directed ratcheting mechanism.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

Depicted in the figures are various aspects of the invention. Elements depicted in one figure may be combined with, or substituted for, one, some, or all of the elements depicted in another figure as desired.

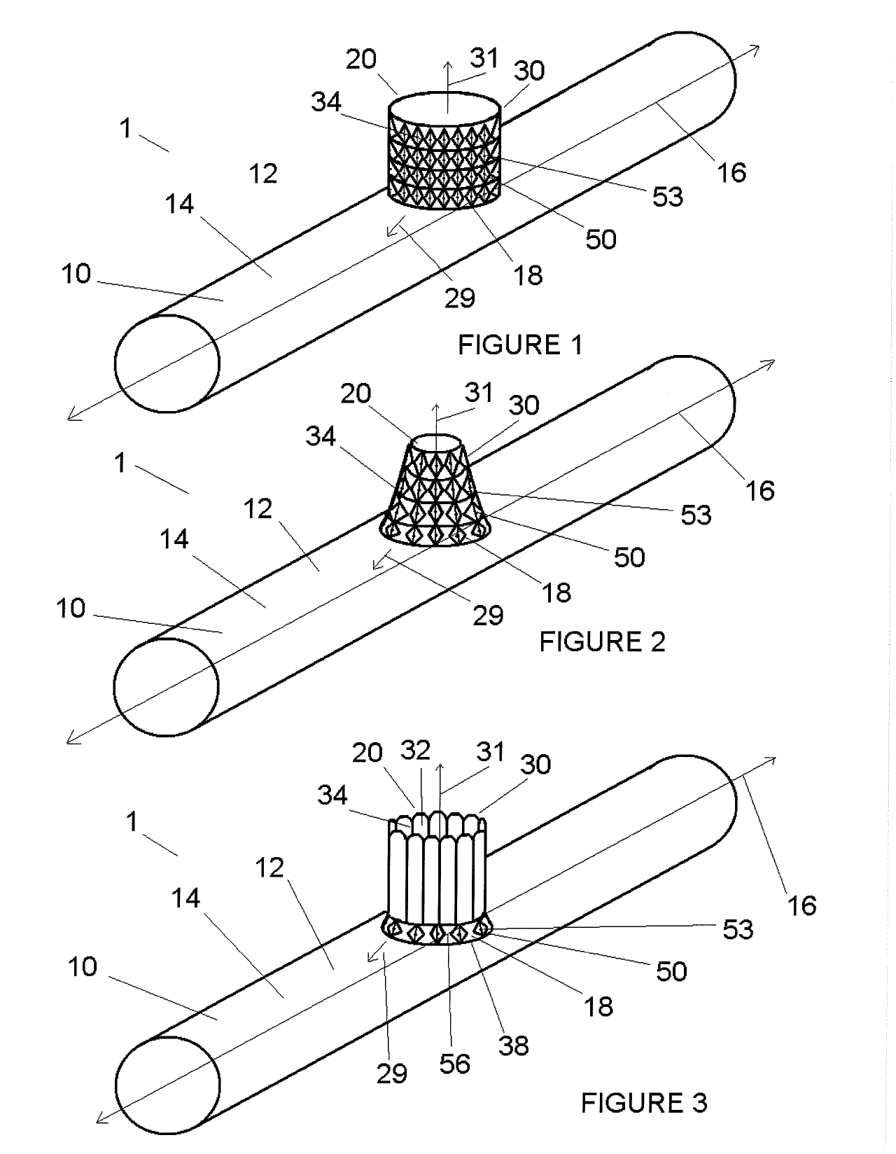

Referring now to FIG. 1, there is shown a bifurcated stent (1) in an expanded state. The stent (1) comprises two portions, a generally tubular main stent body (10) and a bifurcating side branch assembly (30). The side branch assembly (30) is deployed and forms a stent side branch for stenting a body vessel that branches away from the main body vessel that the main stent body stents. The stent (1) as a whole has an expanded state and an unexpanded state (not shown). The main stent body (10) assumes an expanded state using suitable techniques including self expansion, balloon inflation, or by any other method known in the art. When in the expanded state, the stent (1) assumes a greater volume than when in the unexpanded state.

Some or all of the main stent body (10) can be constructed out of one or more metallic materials including but limited to steel, spring steel, stainless steel, titanium, and nitinol and/or out of one or more bioabsorbable materials. For purposes of this application the definition of the term “bioabsorbable” is a metal, polymer or other material or combination thereof which undergoes a chemical or molecular breakdown so as to dissolve, disassociate, or otherwise degrade in the body without ill effect. Such chemical or molecular breakdowns include but are by no means limited to hydrolysis, corrosion, acid corrosion, basic corrosion, oxidation, and any combination thereof. Bioabsorbable materials include but are not limited to materials which degrade when located within the body vessel environments, materials which are absorbed by the vessels they are implanted within or along, and materials which dissolve and while dissolved pass through and out of the body vessels they are implanted within.

Some examples of bioabsorbable materials suitable for the inventive concept include polymers such as bioresorbable polymers. Examples of bioresorbable polymers include, but are not limited to copoly(ether-esters) (e.g. PEO/PLA), cyanoacrylates, poly(amino acids), poly(D,L-lactic acid), poly(glycolic acid), poly(glycolic acid-co-trimethylene carbonate), poly(hydroxybutyrate), poly(hydroxybutyrate-co-valerate), poly(hydroxyvalerate), poly(iminocarbonates), poly(lactide-co-glycolide), poly(L-lactic acid), poly(trimethylenecarbonate), polyalkylene oxalates, polyanhydrides, polycaprolactone, polydioxanone, polyorthoesters, polyphosphazenes, polyphosphoester urethanes, polyphosphoesters, and biomolecules such as fibrin, fibrinogen, cellulose, starch, collagen, hyaluronic acid, etc., and any mixtures or combinations thereof. Other examples of bioabsorbable materials can be found in U.S. Pat. No. 5,358,475 which is hereby incorporated by reference in its entirety. These and other such materials have been referred to as being degradable, biodegradable, biologically degradable, erodable, bioabsorbable, and the like all of which are hereinafter referred to as being bioabsorbable materials.

The main stent body (10) can comprise a number of stent members or struts which together define a first circumferential layer (12). The inner surface of the main stent body (11) faces and defines a first fluid lumen (14). When the stent (1) is in the unexpanded state, at least a portion of the side branch assembly (30) generally lies along or within the first circumferential layer (12) and covers at least a portion of a side opening (18) present in the main stent body (10). In the expanded state, at least a portion of the side branch assembly (30) bends, twists, extends and/or projects away from the first circumferential layer (12) and defines a secondary fluid lumen (34) which is in fluid communication with the first fluid lumen (14). The ostial region of the side branch assembly (30) is engaged to the main stent body. For the purposes of this application, the definition of the term “ostial region” is that portion of the secondary fluid lumen which is located at the junction between the secondary fluid lumen (34) and the main stent body (10).

In at least one embodiment of the inventive concept, the side branch assembly (30) is partially or entirely constructed out of one or more bioabsorbable materials and comprises one or more bio cells (50) with reinforcing locking members (53). When in an unlocked configuration, the bio cells (50) have the needed flexibility and deformity required to properly deploy and position the side branch assembly (30). When locked however, the locking members (53) reinforce the deployed side branch assembly (30) and provide more rigid structural strength.

Referring now to FIG. 3, there is shown at least one embodiment in which two or more bio cells (50) are cooperatively positioned to form a matrix (56) which facilitates the deployment of the side branch assembly (30). In FIG. 3, the bio cell matrix (56) is positioned at the base or ostium (38) of the side branch assembly (30). This bio cell matrix (56) provides the side branch assembly (30) as a whole with needed flexibility during the extension process and subsequently locks it into place when fully deployed.

In at least one embodiment, when in the unlocked configuration, the bio cells (50) are more flexible than petals or petal members (32) of the side branch assembly (30). For purposes of this application the definition of the term “petal” is one or more stent members capable of twisting, bending, pivoting or otherwise opening to define at least a portion of a secondary fluid lumen by opening away from the circumferential layer of the main stent body. The flexible nature of the bio cell matrix (56) and the variety in which they can be shaped facilitates the direction and extension of the side branch assembly (30) at oblique angles relative to the longitudinal axis (16) of the first stent body (10). For the purposes of this application, the definition of term “oblique” is an angle of greater than zero degrees, such as an angle of between about 1 and about 180 degrees and explicitly includes angles of 90 degrees and of about 90 degrees.

Although FIG. 3 illustrates the remainder of the side branch assembly as comprising petal type branching members (32), all known side branch assembly structures known in the art are contemplated by this inventive concept. Some examples of petal type side branch assemblies are described in U.S. Pat. No. 6,835,203, and US Published Patent Application #'s 2005/0102023 and 2004/0138737. The contents of U.S. Pat. No. 6,835,203, and US Published Patent Application #'s 2005/0102023 and 2004/0138737 are hereby incorporated in their entirety by reference. Although FIG. 3 illustrates the bio cell matrix (56) being located at the ostial region (38) of the side branch assembly (30), the bio cell matrix (56) can be located anywhere along the side branch assembly (30).

In at least one embodiment of this inventive concept the side branch formed by the side branch assembly (30) has at least one curve or bend which is formed by the positioning of a bio cell matrix (56) along a portion of the side branch assembly (30). In at least one embodiment, the matrix comprises bio cells (50) arrayed along different locations on the perimeter of the side branch assembly (30) which have different flexibilities, areas, expansive capacities, lengths, and/or resistances to expansive pressures which causes the side branch assembly when deployed to assume an oblique angle relative to the longitudinal axis (16) of the main stent body (10).

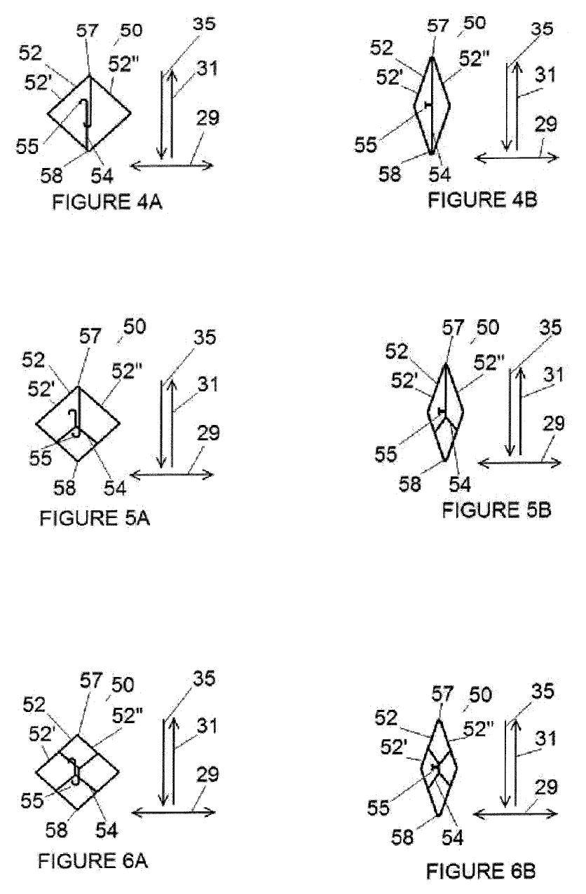

FIGS. 4A, 4B, 5A, 5B, 6A, 6B, 7A, 7B, 8A, and 8B illustrate embodiments of the inventive concept in which a number of different bio cell designs are contemplated. In all of these embodiments the bio cell (50) has an unlocked configuration, (the A alternative of the figures) and a locked configuration, (the B alternative configuration). Every bio cell (50) has a cell wall (52) with at least two oppositely positioned sides, a left side (52′) and a right side (52″). When the side branch assembly is compressed or unexpanded the sides (52′, 52″) are positioned away from each other. In contrast as the side branch assembly deploys it acquires an increase in length provided at least in part by moving the sides of the walls (52′, 52″) together which in turn pushes its ends (corners 57, 58 in FIGS. 4A-8B) farther apart. Because the ends are engaged between other structural components of the side branch assembly (such as rings or struts) moving the walls of the bio cells closer together or farther apart increases or decreases the overall length of the side branch assembly. Bio cells (50) with different flexibilities can be achieved by having walls (52) with different: materials, thicknesses, bending lengths, angles, orientations, or any combination thereof.

When in the unlocked configuration, the bio cell (50) is deformable and the sides of the walls (52) are capable of compression (moving closer) and expansion (moving farther apart) relative to each other. This freedom of motion allows for the flexibility and elasticity needed during the expansion of the side branch assembly (30). Once a sufficient amount of force is applied in a particular direction however, a locking mechanism (53) engages preventing any further compression of the bio cell (50). This situation is desirable when the side branch assembly (30) as a whole has been properly positioned and deployed and deploying force (including but not limited to self expansion tension, stent balloon blister or side balloon pressure, and/or contact with a leader wire) is no longer being applied which requires the deployed side branch assembly (30) to bear the full scaffolding load of the stented body vessel.

FIGS. 4A and 4B illustrate at least one embodiment of this inventive concept. The bio cell (50) is a tetrahedral with a four sided cell wall (52). FIG. 4A illustrates the unlocked configuration where the opposite sides of the cell wall (52′ and 52″) are capable of moving closer or farther apart from each other in response to compressive and expansive forces. Specifically, when this bio cell (50) is in the unlocked state, the walls are capable of moving circumferentially (29) (relative to the first circumferential layer) closer together or farther apart.

As the walls (52) move closer together the most radial corner (57) and the most luminal corner (58) of the bio cell become more distant from each other. For the purposes of this application, the definition of the term “radial” is in a direction oriented away from the side branch opening in the main stent body. Similarly for the purposes of this application, the definition of the term “luminal” is in a direction oriented towards the side branch opening in the main stent body. When the radial and luminal corners (57, 58) are closest together, the bio cell has a shorter radial (31) length which it has when the stent is in the unexpanded state. When these two corners (57, 58) are far apart, the bio cell has an increased radial (31) length which it has when the stent is in the expanded state and the side branch assembly (30) is deployed.

After a sufficient amount of expansive force is applied which moves the two corners (57, 58) apart, the locking mechanism (53) engages, preventing the corners (57, 58) from moving closer again and restraining the bio cell (50) in the locked configuration. In the embodiment illustrated in FIGS. 4A and 4B, the locking mechanism (53) is a pair of oppositely directed hooks (55). The hooks (55) are engaged to shafts (54) which extend from opposite ends of the bio cell (50). In at least one embodiment, the shafts (54) extend from one or both of the most luminal and/or radial corners (57, 58) of the bio cell (50).

When the side branch assembly (30) is not fully deployed, the shafts (54) hold the hooks (55) beyond each other preventing their interaction. Once the bio cell becomes sufficiently radially expanded, the hooks (55) are pulled into each other and prevent the bio cell (50) from assuming the radially shorter unlocked configuration again. In some embodiments such as that illustrated in FIG. 4B, the hooks prevent any contraction which is radially smaller than the locked configuration, but does allow for further radial expansion of the bio cell (50). In other embodiments the locking mechanism is designed to prevent any further radial expansion or contraction once the locked configuration has been assumed by the bio cell (50).

In order to modulate between the need for the bio cell (50) to have sufficient flexibility when in the unexpanded state and to have sufficient rigid scaffolding strength when in the expanded state, a number of embodiments are contemplated by the inventive concept. In FIGS. 5A and 5B, one of the hooks (55) are supported by two shafts (54) and the shafts (54) do not extend from the opposite end of the bio cell (50). The shafts can extend from opposite sides of the cell wall (52) or from any position along the cell wall (52). FIGS. 6A and 6B illustrate both hooks (55) being supported by two or more shafts (54). The number of shafts (54) can be increased or decreased to modulate between increased flexibility of increased scaffolding strength. Similarly the location where the shafts (54) are engaged to the bio cell walls (52) can vary to determine the final shape that the bio cell (50) will be allowed to assume when in the locked configuration.

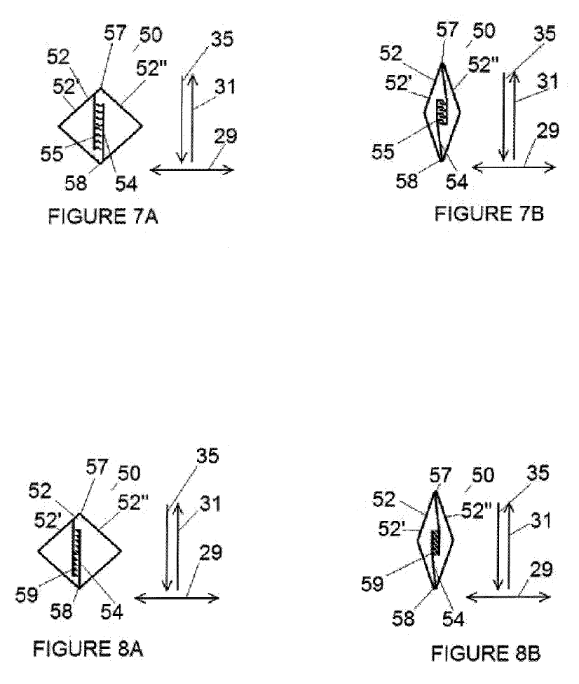

FIGS. 7A and 7B illustrate a bio cell (50) with a locking mechanism comprising multiple locking members. These multiple locking members assure that a minimum radial length is assumed by the bio cell (50) once it is radially expanded to locked configuration but allows for further non-reversible radial expansion beyond this locked configuration. One embodiment of this inventive concept utilizes hooks (55) which are curved to allow one way expansive motion by the interlocked shafts (54). Embodiments contemplated by this inventive concept have either one shaft (54) with multiple hooks (55) or both/all shafts (54) featuring multiple hooks (55). Similarly, embodiments are contemplated in which the hooks of a particular shaft or shafts (54) are concavely curved relative to the radial position the shaft extends them towards (as in FIGS. 4A and 4B) or have a convex curve relative to the radial position the shaft extends them towards (as in FIGS. 7A and 7B).

FIGS. 8A and 8B illustrate a bio cell (50) having a ratchet tooth type locking mechanism (53). Although these ratchet teeth are triangular in shape, any type of ratcheting shape is contemplated by the inventive concept. The ratchet tooth locking mechanism assures that a minimum radial expansion length occurs before the bio cell is radially locked. The ratchet tooth type locking mechanism (53) however allows for further non-reversible radial expansion beyond the locked configuration. The ratchet teeth (59) allow one way expansive motion by the interlocked shafts (54). Embodiments contemplated by this inventive concept have either one shaft (54) with multiple ratchet teeth (59) and the other having a tooth engaging or restraining member, or both shafts (54) featuring one or more ratchet teeth (59).

In addition, the hooks (55) or ratchet teeth (59) of a locking mechanism (53) can allow only radial length changes in the bio cell (50) through the use of shafts so long relative to the lengths of the bio cell walls (52) that any radial movement causes the locking mechanism to engage. In the context of a tetrahedral shaped bio cell (50), this can be accomplished when the length of the shaft (54) is between 75% and 100% (inclusive) of the sum of the lengths of the pair of bio cell (50) wall sides (52) between the most radial and most luminal corners (57, 58) whose combined lengths have the smallest total length. Alternatively the shafts can be so short that the bio cells need to assume near complete radial expansion (meaning that the radially opposite bio cell walls are nearly or are entirely in contact with each other) before the locking mechanism (53) becomes engaged. In the context of a tetrahedral shaped bio cell (50), this can be accomplished when the length of the shaft (54) is between 1% and 25% (inclusive) of the sum of the lengths of the pair of bio cell (50) wall sides (52) between the most radial and most luminal corners (57, 58) whose combined lengths have the smallest total length. In addition, the bio cell can be constructed such that the walls of the bio cell must be stretched before either the locked configuration and/or the maximum of multiple possible locked configurations can be attained.

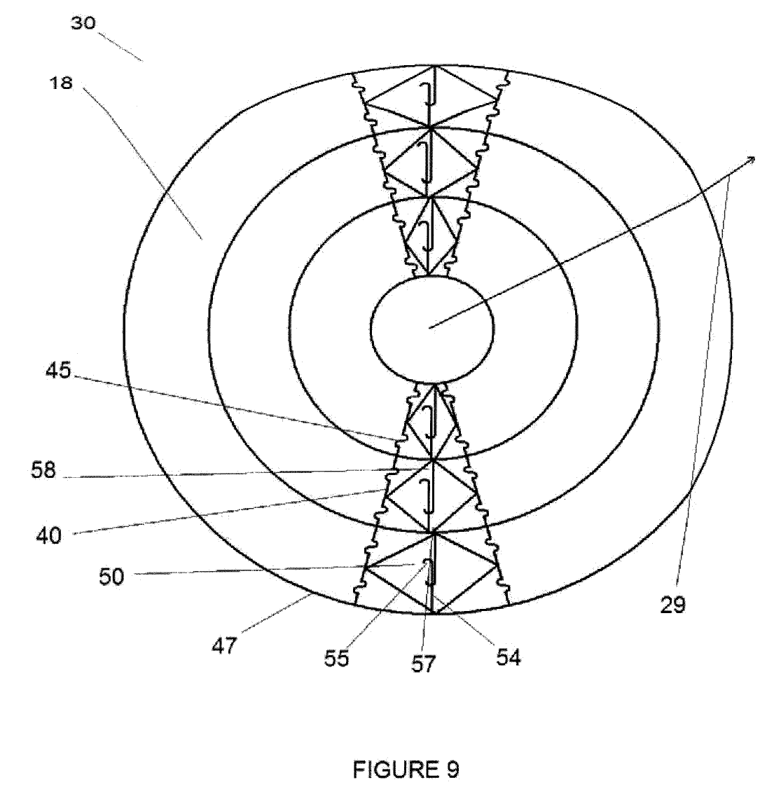

Referring now to FIG. 9 there is shown an unexpanded side branch (30) comprising a number of rings (47) which when expanded will define the walls of the side branch. The rings (47) are moved away from the circumferential layer of the main stent body during deployment. In at least one embodiment, the rings increase their circumference when deployed as described in co-pending commonly owned U.S. patent application Ser. No. 11/300,210, the contents of which is hereby incorporated by reference in its entirety.

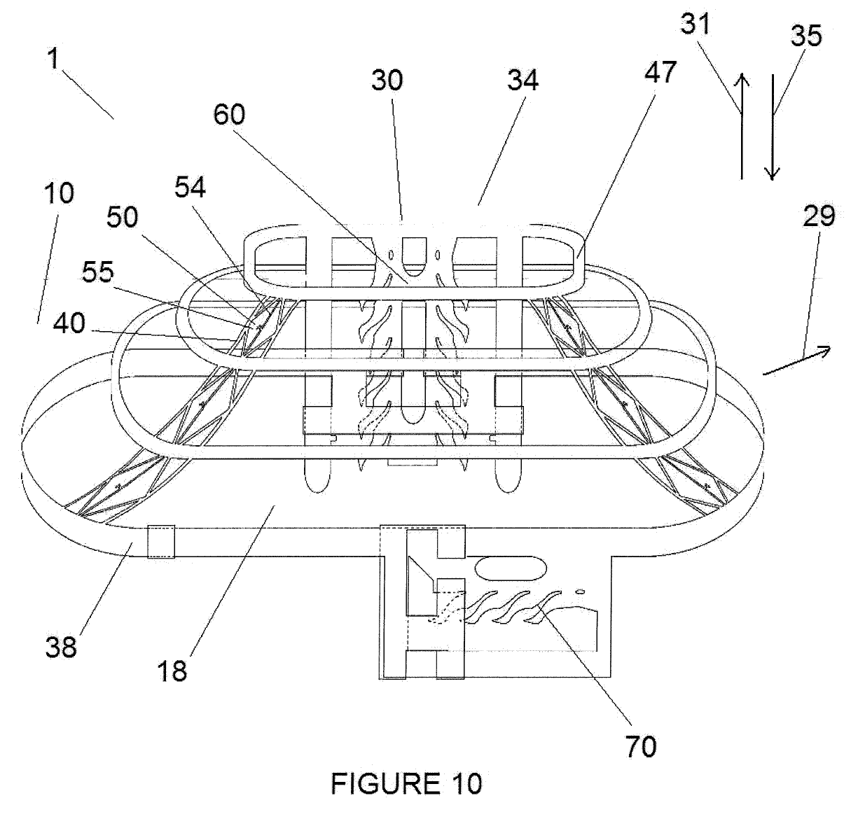

Between at least some of the rings (47) are one or more bio cells (50). Although FIG. 9 illustrates the bio cells (50) as single-hook single-shaft type cells (as described in FIGS. 4A and 4B), any kind of bio cell (50) is contemplated in this inventive concept. As illustrated in FIG. 10, when the side branch assembly (30) is deployed, the bio cells (50) tend to be narrower when measured referenced to a circumferential axis (29) (an axis tangential to the circumferential layer of the main stent body and perpendicular to a radial axis) and tend to be longer when referenced to a radial axis (31). In addition, in FIG. 9, the hooks (55) of the bio cells (50) are disengaged and in FIG. 10 they are interlocked when drawn towards each other during radial expansion of the bio cell (50).

FIG. 9 also illustrates an embodiment in which alongside the bio cells (50) are one or more reinforcing struts (40). A side branch assembly (30) can be further reinforced by these reinforcing struts (40). The reinforcing struts (40) have some structural feature that allows then to contribute to the increase in distance between the rings (47) as they undergo radial deployment. Although FIG. 9 illustrates an embodiment in which the increase in distance is accomplished by the straightening of one or more curved regions (45) in the reinforcing struts (40), other strut designs or other methods of increasing length including the addition of ratcheting lengths to the reinforcing struts (40) is contemplated by this inventive concept. In at least one embodiment, adjacent rings (47) may move closer and farther apart from each other until a minimal radial displacement between two particular rings is achieved.

When deployed, all portions of the perimeters of adjacent rings (47) can be symmetrically equidistant from the respective equivalent perimeter portion of an adjacent ring (47) or the various perimeter portions of adjacent rings (47) can have differing distances from the respective equivalent perimeter portion of an adjacent ring (47). Similarly, any pair of adjacent rings (47) can be positioned closer, equidistant, or farther apart than any other pair of adjacent rings (47). In at least one embodiment, two or more adjacent rings are interconnected by bio cells (50) with dissimilar expansive capacities causing the deployed rings to be positioned at an axis to each other. In at least one embodiment, a curved side branch assembly is achieved by three or more rings having one or more bio cell (50) with a lower expansive capacity on the same side of a ring diameter and one or more bio cell (50) with a higher expansive capacity on the opposite side of that same ring diameter. In addition, a curved side branch assembly is achieved by positioning different numbers of bio cells on opposite sides of one or more rings. In at least one embodiment two or more bio cells having one's luminal corner engaged to another's radial corner are positioned between two adjacent rings.

Referring now to FIG. 10, there is shown an embodiment in which the rings (47) of FIG. 9 have been expanded and moved in a radial direction and define at least a portion of the walls of the side branch. In at least one embodiment, the rings (47) which can be interconnected by bio cells (50) and/or reinforcing struts (40) are positioned such that at least a portion of walls of the side branch are flared. In the context of this application, the definition of the term “flared” is a curved or tapered wall or shape having a concave configuration in which the peak of the arc of the curved/tapering wall faces towards the secondary fluid lumen in the interior of the side branch. The flared shape allows the side branch to match the contoured shape of a branching body vessel which undergoes a rapid drop in circumference.

Also illustrated in FIG. 10 is an embodiment in which the side branch assembly's (30) deployment is at least partially facilitated by a radially directed ratcheting mechanism (60) and in which the side branch opening (18) is widened by a circumferentially directed ratcheting mechanism (70). Each of these ratcheting mechanisms are described in detail below.

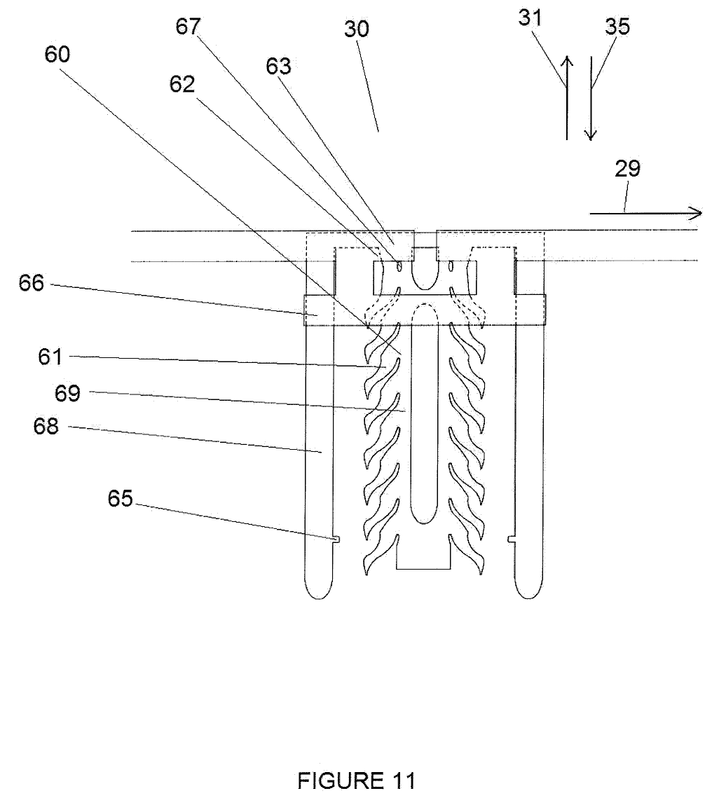

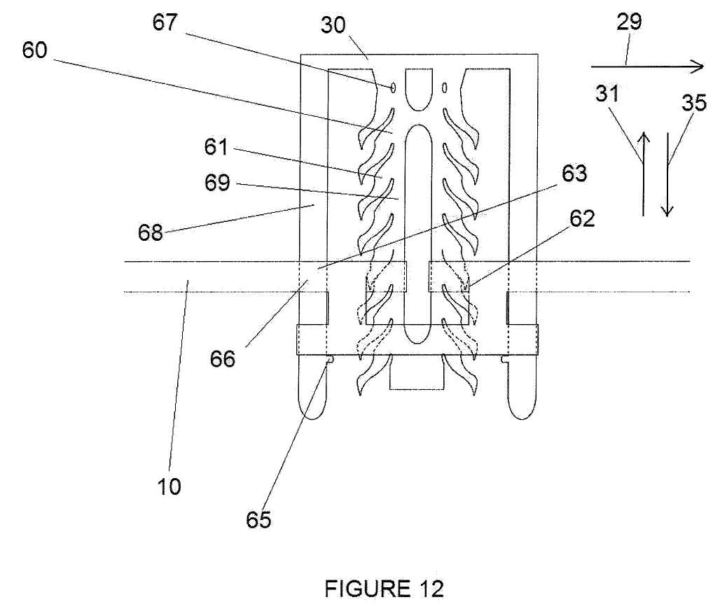

Referring now to FIG. 11 there is shown a radially directed ratcheting mechanism (60). This ratcheting mechanism (60) has one or more teeth members (61) angled such that when positioned against a restraining member (62) the teeth (61) allow for relatively easy motion of the side branch assembly in a radial (31) direction but prevent motion of the side branch assembly (30) in a luminal (35) direction. In at least one embodiment the effectiveness of the ratcheting teeth (61) is enhanced by their at least partial construction out of a bendable material. As illustrated in FIG. 12, while being deployed, as the bendable material teeth (61) pass through a passage port (63) it becomes compressed. This compression can function as a restraining mechanism preventing or inhibiting the premature or unintended deployment of the side branch assembly (30) until the introduction of a radially (31) directed pushing force. In addition, this flexibility allows the restraining member (62) to exert anti-circumferential resistance preventing any unwanted circumferential (29) movement of the side branch assembly.

The ratcheting mechanism (60) may further comprise: a detent (65) to limit the maximum possible radial deployment of the side branch assembly (30), one or more guiding tracks (66) through which a guide rail (68) can pass to better guide the radial passage of the side branch assembly (30), a base member (69) to which the teeth are engaged and/or one or more flex holes (67) in the base member (69) allowing for flexibility in the side branch assembly (30) as a whole. The ratcheting mechanism may be an integrated portion of the wall of the side branch or it may be positioned internally or externally to the walls of the side branch. The teeth (61) may have a blunt or acute shape or be elongated. Similarly the ratchet teeth (61) may be curved as illustrated in FIGS. 10, 11, and 12 or they may be triangular as in FIGS. 8A and 8B. Similarly in addition to or in the place of a passage port (63), there may be two counter-directed arrays of ratchet teeth such as those illustrated in FIGS. 8A and 8B.

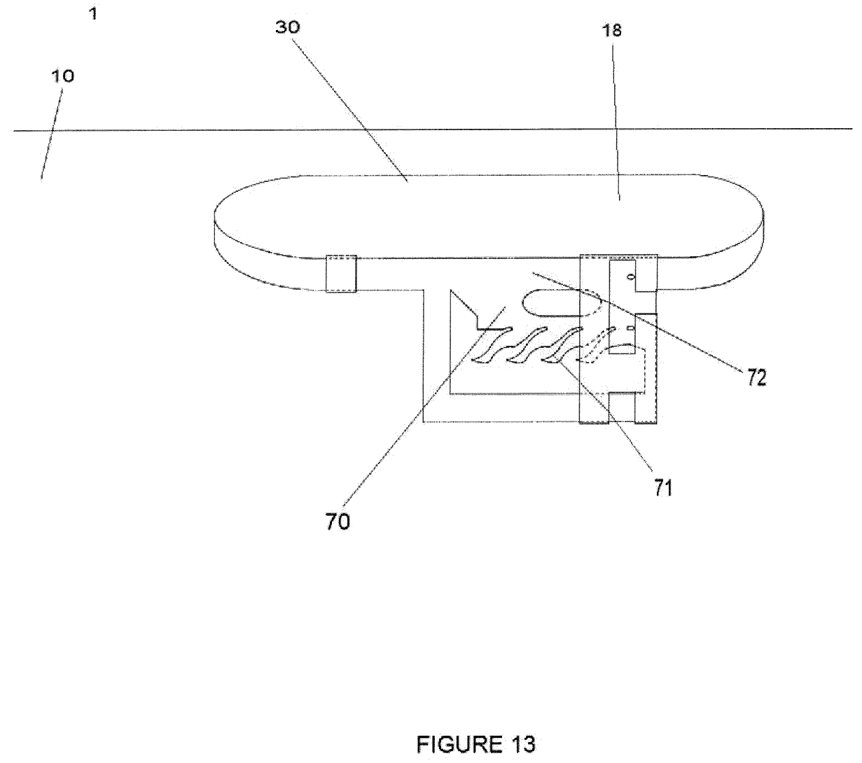

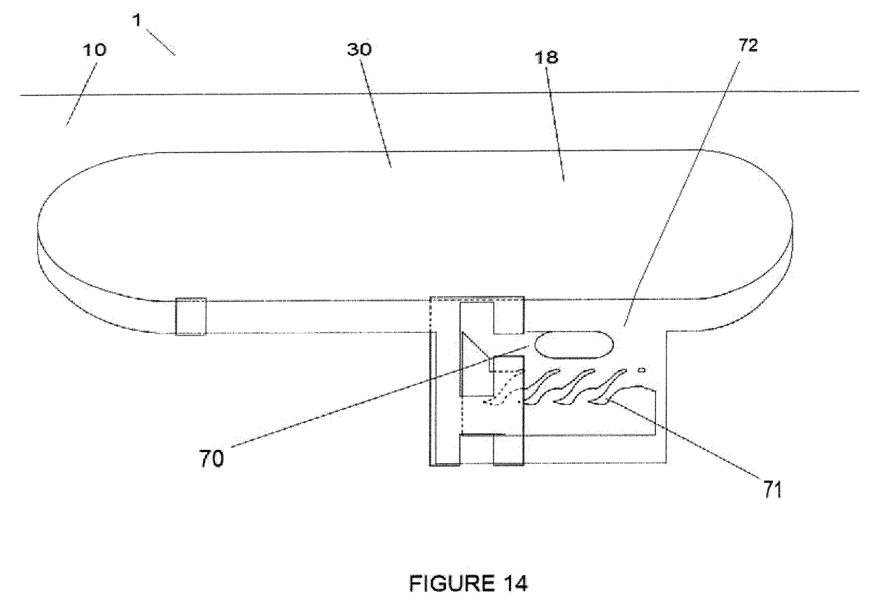

Referring to FIG. 13 there is illustrated a side branch assembly (30) having a circumferentially directed ratcheting mechanism (70). This mechanism allows the side opening (18) of the unexpanded stent to have a greater area when stent (1) is in the expanded state. Such expansion can be used to allow for a wider stent side branch and/or to enhance the flared configuration of the side branch to better match the geometry of the side body vessel. This ratcheting mechanism (70) is similar in design to that of the radially directed ratcheting mechanism. It has one or more ratcheting teeth (71) which allow a widening member (72) to move in one direction but not in an opposite prohibited direction. The allowed movement causes the widening member (72) to move from a circumferentially redundant position (as shown in FIG. 13) to a position where it adds to and increases the overall circumference of at least a portion of the side branch opening (18) (as shown in FIG. 14) or to the circumference of at least a portion of the walls of the side branch.

This ratcheting mechanism (71) can be activated before, during or after the radial deployment of the side branch assembly (30). When the ratcheting mechanism is used after the side branch has been radially extended, it allows the side branch assembly (30) to assume the proper angle and length and is then expanded to accurately and sufficiently fill and scaffold the branching body vessel. Use of this circumferential ratcheting mechanism (71) also allows and enhances the use of athertomes or other bladed or cutting members in conjunction with the deployment of a side branch assembly (30).

In some embodiments the stent, its delivery system, or other portion of an assembly may include one or more areas, bands, coatings, members, etc. that are detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

The therapeutic agent can be at least one or various types of therapeutic agents including but not limited to: at least one restenosis inhibiting agent that comprises drug, polymer and bio-engineered materials or any combination thereof. In addition, the coating can be a therapeutic agent such as at least one drug, or at least one other pharmaceutical product such as non-genetic agents, genetic agents, cellular material, etc. Some examples of suitable non-genetic therapeutic agents include but are not limited to: at least one anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate. It will be appreciated that other types of coating substances, well known to those skilled in the art, can be applied to the stent (1) as well.

This completes the description of the preferred and alternate embodiments of the invention. The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

1. A bifurcated stent having an unexpanded state and an expanded state, the stent comprising:
a generally tubular wall defining a first lumen and a side branch deploying portion constructed of at least one bioabsorbable material engaged to the generally tubular wall,
when in the expanded state, the side branch deploying portion extends obliquely outward from the generally tubular wall and forms a side branch which defines a second lumen in fluid communication with the first lumen,
the side branch deploying portion comprises one or more bio cells, said bio cells have a perimeter which defines an opening in the side branch deploying portion, a luminal end at the portion of the perimeter closest to the tubular wall, a radial end at the portion of the perimeter farthest from the tubular wall, and a locking mechanism, said locking mechanism imposing on the bio cell locked and unlocked configurations,
when the bio cell is in the locked configuration, the luminal and radial ends are restrained from moving closer to each other.

2. The stent of claim 1 in which the side branch assembly further comprises one or more petal members having a free end and having an engaged end engaged to another component of the stent,
when the stent is in the unexpanded configuration the petal members extend over an opening in the generally tubular member of the stent,
when the stent is in the expanded configuration the engaged end bends obliquely relative to opening in the generally tubular member of the stent which moves the petal as a whole to form at least a portion of the side branch wall.

3. The stent of claim 1 in which there is a matrix of bio cells comprising a plurality of bio cells positioned such that the matrix encircles at least a portion of the side branch.

4. The stent of claim 3 in which the bio cell matrix extends along that portion of the side branch located where the tubular wall and the side branch are engaged to each other.

5. The stent of claim 3 in which at least a portion of the bio cell matrix is arranged according to a circular pattern and in which at least two bio cells on opposite sides of the circular pattern have different expansive capacities.

6. The stent of claim 3 in which at least a portion of the bio cell matrix is arranged according to a circular pattern and in which the walls of at least two bio cells on opposite sides of the circular pattern have perimeters with different overall lengths.

7. The stent of claim 1 in which the locking mechanism comprises at least two shafts each of which extends between a position along the perimeter of the bio cell and an engaging member, each of the engaging members become engaged to each other when the shafts are moved into contact with each other as the stent enters into the expanded state.

8. The stent of claim 7 in which each of the engaging members comprise at least one hook positioned to retain each other when pulled close to each other.

9. The stent of claim 8 in which the hooks arc concave relative to the position along the perimeter of the bio cell from which the shaft the hook is along extends from.

10. The stent of claim 8 in which the hooks arc convex relative to the position along the perimeter of the bio cell from which the shaft the hook is along extends from.

11. The stent of claim 7 in which there are multiple engaging members along each shaft, when any two engaging members of the shafts become mutually engaged the engaging members permit deformation of the bio cell perimeter in a radial direction but prevent further deformation of the bio cell in a luminal direction.

12. The stent of claim 7 in which the engaging members are oppositely oriented triangular members.

13. The stent of claim 7 in which the bio cell is tetrahedral in shape and comprises four interconnected walls, two right sided walls and two left sided walls, the right sided walls being an outer right wall, and an inner right wall, the left sided walls being an outer left wall, and an inner left wall.

14. The stent of claim 7 in which at least one of the shafts has a length within the range of at least 1% and no greater than 25% of the lowest sum of the lengths of two similarly sided walls.

15. The stent of claim 7 in which at least one of the shafts has a length within the range of at least 75% and no greater than 100% of the lowest sum of the lengths of two similarly sided walls.

16. The stent of claim 1 in which at least some portion of side branch assembly is coated with at least one therapeutic drug.

17. The stent of claim 1 further comprising two or more immediately adjacent bio cells extending between a first stent member closer to the center of the side branch deploying portion of the stent and a second stent member farther away from the center of the side branch deploying portion of the stent, between the at least two immediately adjacent bio cells extending from the first stent member to the second stent member is a reinforcing strut, the reinforcing strut having at least one linear portion and at least one curved portion, the curved portion capable of at least partially straightening when the distance between the first and second stent members increases as the stent transitions form the unexpanded state to the expanded state.

18. The stent of claim 1 in which first and second stent members are rings which when the stent is in the unexpanded state, are concentrically positioned relative to each other and extend over an opening in the generally tubular stent body,
when the stent is in the expanded configuration, the rings are positioned serially away from the opening in the generally tubular stent body and form at least a portion of the side branch.

19. The stent of claim 1 in which the side branch deploying portion of the stent further comprises a ratcheting mechanism, the ratcheting mechanism comprising a base member from which two or more teeth members extend, the teeth members have at least one angled surface which is capable of only one way passage relative to a restraining member, the one way passage facilitating the movement of at least some of the side branch deploying portion away from the generally tubular body of the stent, when the side branch is being deployed, the restraining member retains its position relative to the generally tubular body of the stent.

20. The stent of claim 1 in which the side branch has a flared configuration.
